(19) United States
(12) Patent Application Publication
(10) Pub. No.: US 2020/0253729 A1
(43) Pub. Date: Aug. 13, 2020

(54) UNIVERSAL HEART VALVE DEVICE

(71) Applicant: W. L. Gore & Associates, Inc., Newark, DE (US)

(72) Inventor: John LASCHINGER, Lutherville, MD (US)

(21) Appl. No.: 16/272,076

(22) Filed: Feb. 11, 2019

(51) Int. Cl. A61F 2/24

(52) U.S. Cl. CPC A61F 2/2418; A61F 2/2433; A61F 2/2445; A61F 2/2436; A61F 2230/005; A61F 2250/0029; A61F 2/2496; A61F 2220/0016; A61F 2250/0048

(57) ABSTRACT

Embodiments of universal self-anchoring prosthetic valves for multi-position transcatheter or surgical implantation within any diseased or malfunctioning native heart valve are provided. An exemplary embodiment of a prosthetic valve includes a radially compressible and self-expanding central core housing a prosthetic valve, with compressible and flexible memory-shaped woven wire anchoring discs at the inflow and outflow ends. Specific design properties allow the prosthesis to be conformable, self-centering and flipped for multiple appropriate physiologic implant orientations. Expansive transverse radial force exerted by the central core, and memory-shape induced directional forces exerted by flexible inflow and outflow discs, capture and compress native peri-annular and leaflet tissues to anchor the prosthesis. Tissue facing frame surfaces may include small tines to enhance anchoring. The prosthetic valve frames may include various biomaterials or polymers as linings, coatings or coverings to enhance sealing. Methods and devices for delivering and implanting the valve based on access are described.

### BACKGROUND

Replacement heart valves have been utilized since as early as 1960. Prior to the last decade, heart valve devices were designed for open surgical valve implantation requiring prolonged cardiopulmonary bypass, stoppage of the heart and direct suturing after appropriate debridement of native or diseased tissue to ensure effective, long-term fixation to the remaining native annular and or valvular tissue (surgical valves). As a result, open surgical procedures inherently carry a significant amount of risk as well as a more burdensome recovery period related to the need for highly invasive open surgical access, cardiopulmonary bypass, and stoppage of the heart. Beginning in the early part of this century, bioprosthetic heart valves designed for specific individual positions within the heart have been developed that are designed for percutaneous implantation using transcatheter techniques. Based on implant position within the heart, various transcatheter heart valve devices have been developed that rely on radial force or anatomic methods of fixation necessary to allow transcatheter implantation. Though less invasive, these devices may result in a higher incidence of paravalvular leak which, if moderate or greater, may require additional procedures or result in higher long-term mortality. Specific pressure dependent deployment and fixation methods for transcatheter valves, especially those in the aortic position, may also lead to a high incidence of need for permanent pacemakers. Whether conventional surgical and transcatheter valve devices are used, each replacement valve is designed for a specific position within the heart (i.e. ventriculo-aortic valves [V-A valves]—aortic or pulmonary positions; atrio-ventricular valves [A-V valves]—mitral or tricuspid positions), the planned route of implantation access (i.e. stiff/semi-flexible frame and rigid sewing ring for open surgical access vs. collapsible transcatheter frame for peripheral access), and the planned method of fixation (suture vs. sutureless or anatomic). Therefore, conventional valves cannot be positioned universally.

Current transcatheter heart valves (THVs) are designed for one mode of access (transcatheter), one position within the heart (e.g. aortic, mitral, tricuspid or pulmonic), and for one pathology (i.e. either calcific stenotic lesions or lesions resulting in insufficiency)—one access, one position, one pathology. This is primarily dictated by valve design, principally related to the anchoring mechanism needed for the insertion location and the underlying pathology effects on the valve leaflets and annulus. Due to constraints related to designed valve housing and anchoring mechanisms: a valve designed for the Ventriculo-Arterial (V-A) position (aortic or pulmonic) cannot be reliably used (and is not indicated for use) in the native Atrio-Ventricular (A-V) position (mitral or tricuspid); a valve designed for anchoring within a calcific stenotic native valve cannot be reliably used (and is not indicated for use) for lesions resulting in primary insufficiency of the native valve; in addition all currently designed aortic THVs have a clinically significant incidence of paravalvular leak. Furthermore, current surgical heart valves (SHVs) require placement under direct vision during open heart surgery using full cardiopulmonary bypass and cardioplegic or fibrillatory arrest. Surgery times are prolonged by the need to prepare the valve tissue and annulus (e.g. resection or debridement of tissue) to accept and then place the multiple sutures needed for insertion and anchoring of the valve. Valve sewing rings are designed for specific positions within the heart but the risk of paravalvular leak remains. Under specific circumstances, clinical need may dictate use of these valves in a ‘flipped’ orientation (e.g. aortic valve flipped and used in mitral valve position where appropriate size mitral prosthesis is not available) but this is primarily used for off-label pediatric applications. Current surgical valves cannot be placed via closed heart surgical procedures. Lastly, bioprosthetic valves, regardless of position (V-A or A-V) or type (THV or SHV) are unidirectional tri-leaflet valves made of similar materials (e.g. bovine pericardium) differing primarily in mounting orientation within the valve frame and anchor housing to allow one-way blood flow consistent with the planned position of use.

It may therefore be desirable to have a universal heart valve device that can be manufactured in a predetermined range of sizes (i.e. every size) and deployed using a minimally invasive transcatheter approach as well as “snap in” open or closed heart surgical insertion (i.e. every method and route of access). It also may be desired to have a single universal valve device that, in addition to the above, can be deployed in the appropriate orientation in all heart valve positions and maintain long-term functionality and durability (i.e. every position). Finally, it may also be desirable for this valve to, in addition to the above, have an anchoring mechanism that may result in reliable fixation on any underlying valve and annular tissue regardless of pathology (i.e. every pathology).

### SUMMARY

According to an exemplary embodiment, a sutureless, self-anchoring universal heart valve device in various sizes may be provided. The universal heart valve device may have a collapsible or crimpable frame including a hollow annular-sized central core from which a bioprosthetic (or biocompatible polymeric) replacement valve may be suspended. The device may further include a flexible skirt disc extending from an in-flow side of the annular central core and a flexible out-flow skirt disc extending from an outflow side of the annular central core. The inflow and outflow ends of the device may be oriented according to the direction of normal physiologic blood flow (i.e. in the axis of flow) such that the device may be placed in any position of the heart. Furthermore, the device may have a plurality of tines disposed on a tissue-facing outer surface of at least one of the central core, the in-flow skirt disc, and the outflow skirt disc to aid in multi-position, multi-pathology anchoring. Lastly, the in-flow skirt disc may have a larger diameter than the outflow skirt disc, and either of the skirts and/or the central core may be coated, covered or lined with biologic or biocompatible prosthetic materials (i.e. biomaterials) to improve sealing and healing, and/or to minimize the risks of paravalvular leaks.

According to another exemplary embodiment, a method of deploying a universal heart valve device may be provided. The method may include determining a position for the device within a native heart valve, determining a method of access to the native heart valve to be replaced, determining a route of access for deployment of the device, measuring the native valve annulus and paravalvular dimensions, and selecting an appropriately sized device for the native valve to be replaced. The device may then be mounted, loaded, and crimped in a steerable deployment catheter in a direction appropriate for the location of use, method and route of access, and the direction in which the valve is crossed. The device may be steered within the deployment catheter to align with the native annulus and valve tissue. The device may further be positioned and deployed in an order according to the site and direction of access, and may promote conforming to native tissues, self-alignment, and self-centering along the axis of flow within annular and native valve tissues.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows a perspective view of an exemplary embodiment of a universal heart valve device;

FIG. 2 shows a side elevation view of an exemplary embodiment of a universal heart valve device;

FIG. 3 shows a perspective view of an exemplary embodiment of a universal heart valve device;

FIG. 4 shows an exemplary embodiment of a universal heart valve device;

FIG. 5 shows an exemplary embodiment of a universal heart valve device;

FIG. 6 shows an exemplary embodiment of a universal heart valve device;

FIG. 7 shows an exemplary embodiment of a universal heart valve device;

FIG. 8 shows an exemplary embodiment of a universal heart valve device;

FIG. 8A shows an exemplary embodiment of a universal heart valve device;

FIG. 9 shows an exemplary embodiment of a universal heart valve device in a Ventriculo-Aortic position;

FIG. 10 shows an exemplary embodiment of a universal heart valve device in an Atrio-Ventricular position;

FIG. 11 shows an exemplary embodiment of a universal heart valve device and deployment catheter;

FIG. 12 shows an exemplary embodiment of a universal heart valve device and deployment catheter;

FIG. 13 shows exemplary embodiments of a universal heart valve device deployed at desired positions in a heart;

FIG. 14 shows an exemplary embodiment of a biomaterial coating on a wire prosthetic device;

FIG. 15 shows an exemplary embodiment of a biomaterial lining in a wire prosthetic device;

FIG. 16 shows an exemplary embodiment of a biomaterial covering on a wire prosthetic device;

FIG. 17A shows a conventional replacement valve device;

FIG. 17B shows a conventional replacement valve device;

FIG. 17C shows a conventional replacement valve device;

FIG. 18A shows an exemplary embodiment of a universal heart valve device;

FIG. 18B shows an exemplary embodiment of a universal heart valve device; and

FIG. 18C shows an exemplary embodiment of a universal heart valve device.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

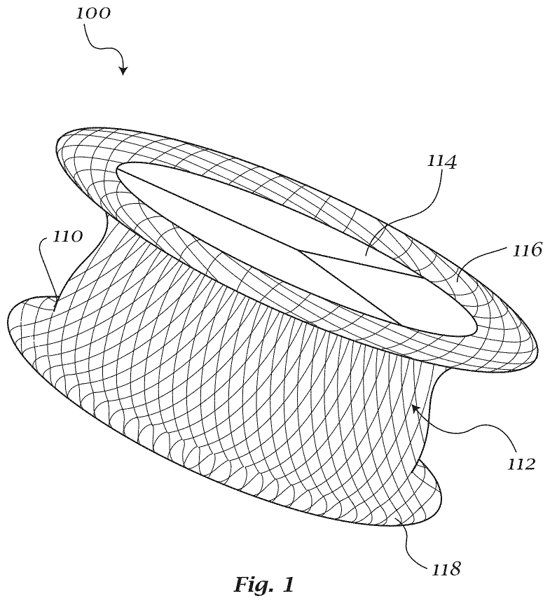

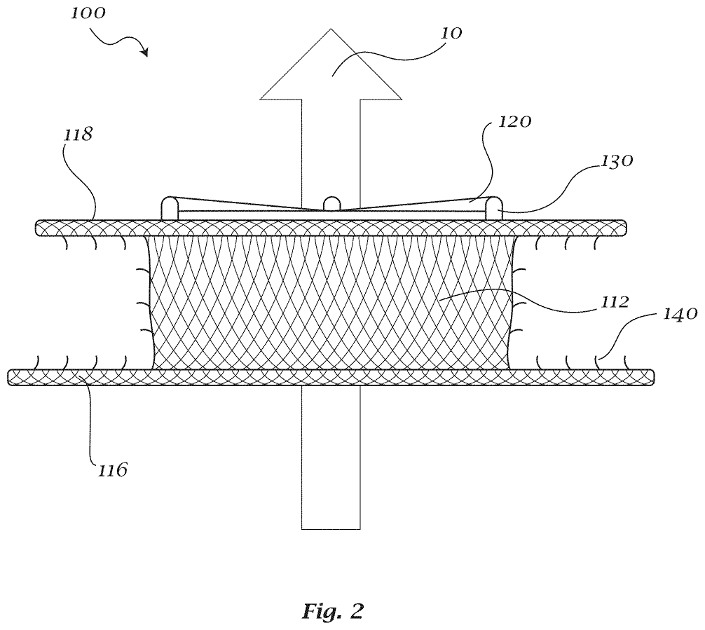

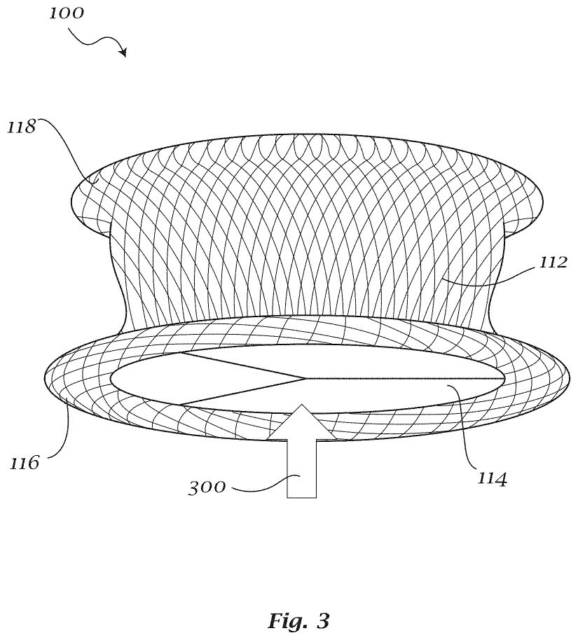

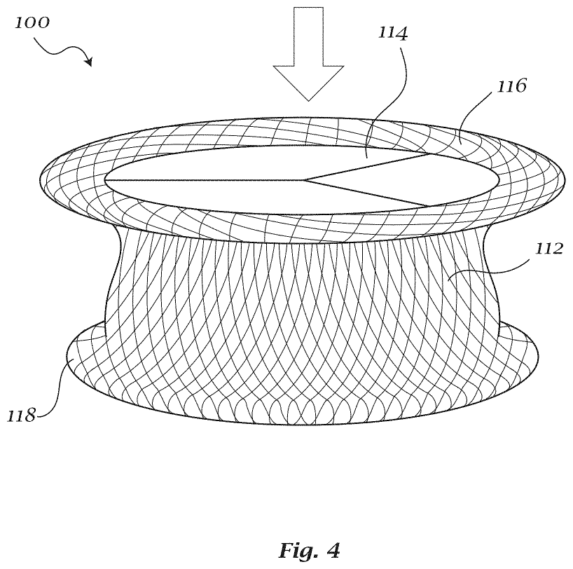

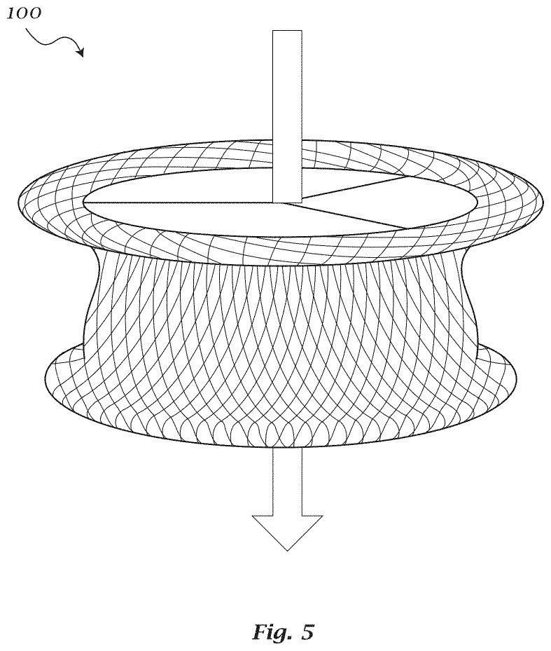

Now referring to exemplary FIG. 1, a universal heart valve device 100 may be provided. Universal heart valve device 100 may facilitate transcatheter or direct surgical insertion of replacement heart valves. The unique design of device 100 may be available in a plurality of sizes to allow for universal use in all positions and all pathologies regardless of the method or route of access. More specifically, a single universal valve device 100 may be mounted, crimped and deployed in Ventriculo-Arterial (V-A) orientation for Aortic valve or Pulmonary valve replacement (FIGS. 2 and 3), and may be flipped (mounted, crimped and deployed) in a an Atrio-Ventricular (A-V) orientation for Mitral or Tricuspid valve replacement (FIGS. 4 and 5). Depending on positioning within the heart and the method and route of access, the universal valve device 100 may be deployed in each orientation, without structural modification, by altering the direction in which the valve is mounted and crimped on the deployment catheter(s). Access routes for the universal valve device 100 may include all variations of direct surgical vision open heart routes, image guided percutaneous transcatheter arterial or venous routes, and indirect surgical closed heart image guided insertion including but not limited to per-arterial, per-venous, per-ventricular and per-atrial access routes. For the purposes of this application, a universal heart valve device (or “universal valve device” or “the device”) may refer to a self-anchoring collapsible frame bioprosthetic (or biocompatible polymeric) heart valve that may be conforming, self-aligning, self-centering and self-sealing, and which may be used in any pathology in any position of the heart using any method or route of access for implantation. Device 100 may allow for use with all pathologies, including calcific stenotic lesions and lesions resulting in insufficiency, due to its unique anchoring system, which may include three levels. Device 100 may not be dependent on the presence of calcium (stenotic lesions) or tissue hooking mechanisms (insufficiency lesions) to work. Additionally, device 100 may be utilized via all access methods and routes, including all image guided transcatheter access vessels and routes, direct visual access at open heart surgery, and image guided closed heart surgical access (e.g. transapical, peri-ventricular, per-atrial, per-aortic or per-pulmonic) during surgical procedures. As referenced herein, valve device 100 may be any of various manufactured sizes, including the valve size and frame size to ensure proper fit and physiologic function in any location.

Universal heart valve device 100 may have a frame 110. The frame material may be collapsible and crimpable to allow device 100 to be sheathed and deployed to a desired location, shape and orientation. Metallic components of frame 110 may have a composition and variable cell structure (or weave pattern) designed to maximize flexibility and shape memory of the central core 112 and discs 116, 118 in order to sustain an in-round central core which may be necessary to result in self-centering and to maintain optimal physiologic valve function and maximize anatomic conformity (optimize fixation, sealing and healing) to para-annular and valvular tissues. Frame 110 may be a self-expanding memory-shape frame and may optionally have an open or closed cell design of one or 2 layers. According to at least one exemplary embodiment, frame 110 may be a wire mesh or woven wire design. The material of frame 110 may have programmable properties, such as shape memory, as would be understood by a person having ordinary skill in the art. For example, frame 110 may be flexible and easily manipulated, but may revert to a programmed or otherwise formed natural resting shape, which may promote anchoring and deployment in a desired position within a patient. The programed shape and material properties may be resistant to undesired movement or deformation from heart functioning when in a deployed state, as would be understood by a person having ordinary skill in the art. According to an exemplary embodiment, frame 110 may be made of a material exhibiting these properties, such as Nitinol. Frame 110 may be made of other materials having strong shape memory and super-elasticity properties, as would be understood by a person having ordinary skill in the art. According to some alternative exemplary embodiments, frame 100 may be made of stiffer materials that would require balloon dilation to achieve a final shape and fixation. In such alternative embodiments, frame 110 may have a foreshortened, wide hourglass shape.

Frame 110 may further include a central orifice 114, which may be defined by a central core portion 112 of frame 110. When deployed, central core 112 of frame 110 may exert sufficient radial force to fully expand a valve and maintain circularity of the frame when deployed. Central core 112 may exert or maintain sufficient outward radial force to sustain full “in-round” valve deployment, as opposed to oval or out-of-round deployment, and also promote self-centering and secure sutureless fixation to native valve and para-annular tissues. Frame 110 may have skirts 116, 118 disposed on each end of the central core 112. Central core 112 may be substantially cylindrical and may vary in length according to valve size and/or suspension height of the valve tissue within the cylinder. Ring shaped and skirts 116, 118 may project outward from central core 112 in a disk-like manner. In an exemplary embodiment, a flexible in-flow skirt 116 may be disposed on the in-flow side of central core 112 and a flexible out-flow skirt 118 may be disposed on the out-flow side of central core 112. According to some embodiments, in-flow skirt 116 may have a larger diameter, which may provide better conforming, anchoring and sealing due to shape memory, the forces of blood flow, anatomic considerations, differential blood pressures and functioning of the heart. The dual in-flow skirt 116 and outflow skirt 118 may allow for deployment in multiple positions by flipping the device mounting and deployment orientation depending on access method and route and the position and desired direction of flow (ventriculo-arterial or atrio-ventricular).

A replacement heart valve may be supported by device 100 and may be integrally connected to frame 110. In some alternative exemplary embodiments, a replacement valve may be directly attached-to or suspended from the inner central core, or may have an independent valve frame which may be affixed within or suspended from an inner portion of frame 110, resulting in a multi-piece device as would be understood by a person having ordinary skill in the art. In multi-piece embodiments, individual pieces may optionally be deployed, interchanged, and/or replaced in unison or separately.

According to some alternative exemplary embodiments, a valve device may have an hourglass shape as depicted in FIG. 1; however, the device may only have a flexible and conformable inflow disc as depicted in FIG. 16. The outflow end in such embodiments may exhibit properties similar to the central core, such as exerting radial force and being substantially inflexible.

Now referring to exemplary FIG. 2, a valve device 100 may be shown in a resting state. As shown, the blood flow direction through the central orifice may be represented by arrow 10. According to some exemplary embodiments, in-flow skirt 116 may have a diameter greater than out-flow skirt 118. The larger diameter of in-flow skirt 116 may provide additional surface area for contacting, conforming to, and gripping native tissue on an in-flow side (i.e. proximal to the annulus in the direction of flow), which may maintain the valve device's placement against forces from blood flow, as would be understood by a person having ordinary skill in the art. In addition to better anchoring, the larger, flexible in-flow skirt 116 may provide for better conformity and sealing, preventing blood leakage around the valve device 100 reducing or preventing paravalvular leak. The frame 110, including at least portions of skirt discs 116, 118 and/or central core 112 may be coated, covered or lined with materials designed to optimize sealing and promote healing, growth, and integration with natural tissue. In some exemplary embodiments, this material may be PTFE or other polymeric or biologic materials such as, but not limited to, Dacron™ (polyethylene terephthalate) and ovine or bovine pericardium. The coating, covering or lining may be flexible such that the underlying flexibility of the frame is not impacted and retains the ability to conform with anatomy and promote anchoring. A combination of coatings, coverings and/or linings may promote an effective seal at up to 3 levels (inflow disc, central core, and outflow disc) providing secure anchoring (fixation, sealing and healing) and providing important deterrents for minimizing or preventing the risk of paravalvular leaks (PVL).

Universal heart valve device 100 may have a replacement valve 120 affixed to frame 110 within central orifice 114 of central core 112 such that blood flow may traverse the central orifice 114 unidirectionally. An exemplary replacement valve 120 may be a central trileaflet valve and may be a bioprosthetic or other artificial replacement valve appropriately sized for the patient and the anatomy. Bioprosthetic replacement valves may include, for example, valves made from ovine or bovine pericardium, other animal or human tissues or their derivatives (e.g. extracellular matrix, etc.), or biocompatible polymeric tissues (e.g. PTFE or ePTFE and their derivatives, etc.). Replacement valve 120 may be oriented centrally within frame 110 and may be oriented to open unidirectionally according to the direction of desired blood flow 10 and close completely with negligible central valvular leak. Anti-calcification treatments may be applied to leaflet tissues. Furthermore, the suspension height/depth within central orifice 114 may be altered to optimize hemodynamic performance and valve washing and the length of the central core 112 may vary as needed to accommodate changes in valve suspension/height/depth.

Valve device 100 may have valve commissure posts 130, which may facilitate mounting or suspension of a replacement valve 120 to frame 110 within the central orifice 114 of central core 112. Commissure posts 130 may be affixed to frame 110 and may be disposed within central orifice 114 to project beyond outflow skirt 118. In exemplary trileaflet embodiments, there may be three commissure posts 130, which may each secure a portion of at least two valve leaflets, as would be understood by a person having ordinary skill in the art. Valve leaflets may be affixed to commissure posts using known securing techniques, including suturing, as would be understood by a person having ordinary skill in the art. In some alternative exemplary embodiments, valve leaflets may be affixed directly to frame 110 or an independent valve frame affixable to frame 110 within the central orifice 114 of central core 112.

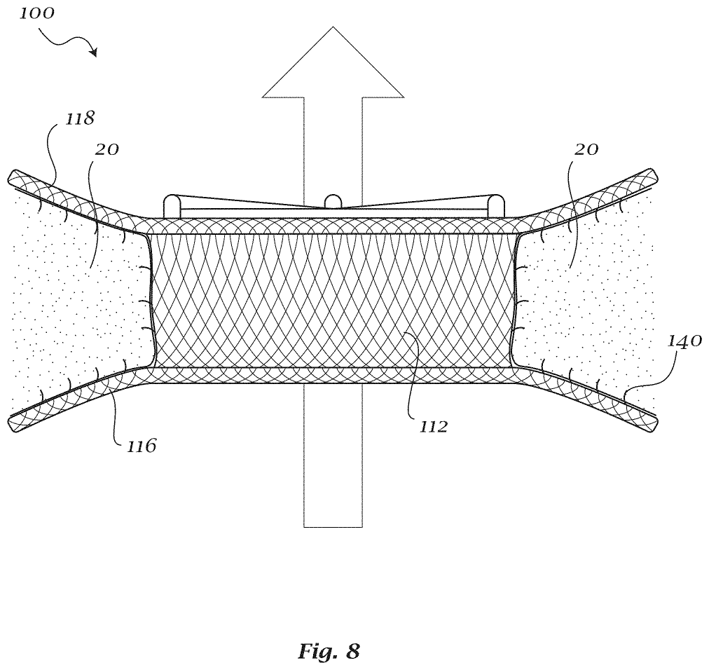

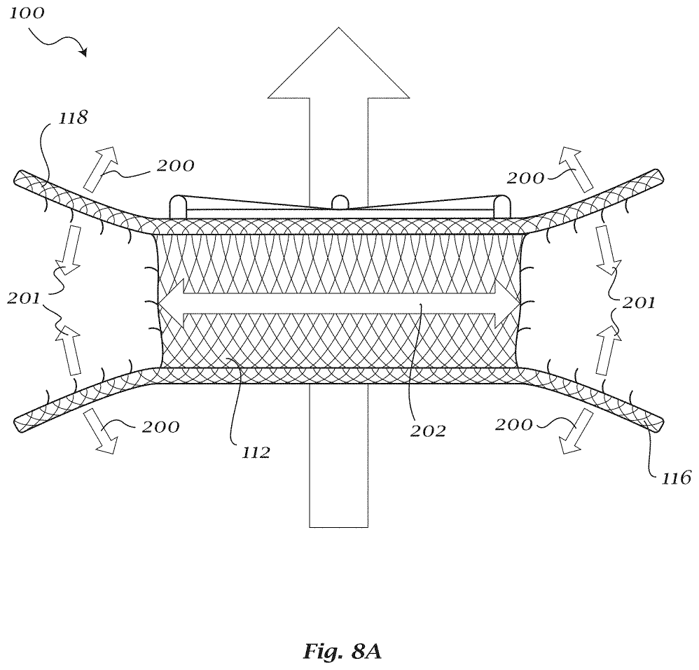

Frame 110 may further include a plurality of barbs or tines 140. Exemplary FIG. 8 shows tines disposed from tissue-facing surfaces of frame 110 adjacent to native tissue 20 when deployed. Tines 140 may be disposed on tissue-facing surfaces of frame 110 such that tines 140 project at various angles and engage surfaces of the native tissue 20 adjacent to the valve and disc frame to facilitate and aid secure valve fixation. Tines 140 may enhance fixation, anchoring, and tissue engagement by penetrating and/or friction. According to some exemplary embodiments, at least some of tines 140 may project at particular angles to resist movement in particular directions, including the direction of blood flow. Tines 140 may optionally project in the same direction and angle or in different directions and angles, as would be understood by a person having ordinary skill in the art. Tines 140 may optionally be disposed on one or more of the tissue-facing surfaces of central core 112, the in-flow skirt 116, and the out-flow skirt 118. Tines 140 may optionally be the same material as frame 110 and according to an exemplary embodiment may be up to about 1 mm in length and oriented toward adjacent native tissues 20. Tines 140 may be oriented such that physiologic forces (pressures and blood flow) would promote anchoring of the tines 140 in native tissue 20. The expansion force of frame 110 at central core 112, rebound force of the flexible discs 116 and 118, and gripping force of tines 140 may work in combination to allow device 100 to conform to and grip native tissue to sufficiently and securely anchor device 100 without the need for sutures or a specific underlying pathology.

According to exemplary FIG. 3, a valve device 100 may be shown in a Ventriculo-Arterial (V-A) orientation for replacement of Aortic and Pulmonary Valves. In transcatheter approaches to the aortic valve and direct open surgical approaches to the aortic and pulmonic valves, an appropriately sized device 100 may be inserted using a retrograde crossing approach through the native V-A valve as would be understood by a person having ordinary skill in the art. This may be accomplished using access through a percutaneous femoral artery or other peripheral artery transcatheter access system (Aortic valve, AoV), or under direct vision at open heart surgery via incisions in the aorta (AoV) or pulmonary artery (pulmonary valve, PV). Alternatively, an antegrade crossing insertion approach may be used for either transcatheter pulmonic valve replacement using an appropriately sized device, as would be understood by a person having ordinary skill in the art. This may be accomplished using access through the femoral or other peripheral venous access system, or for closed heart surgical access using trans-apical (AoV) or per-ventricular (AoV or PV) approaches. The decision for use of a retrograde or antegrade native V-A valve crossing deployment approach may determine the direction in which the appropriately sized device may be mounted and crimped on the deployment catheter and the deployment sequence. For retrograde native valve crossing deployments, the ventricular end in-flow skirt 116 may be deployed first, followed sequentially by deployment of the central core 112 and valve 114, and subsequently the arterial end outflow skirt 118. For antegrade native valve crossing deployment, the valve may be mounted and crimped in the opposite direction on the deployment catheter, and a reverse deployment sequence may be used (i.e. outflow disc deployed first).

As shown in exemplary FIG. 4, a valve device 100 may be shown in an Atrio-Ventricular (A-V) orientation for replacement of Mitral and Tricuspid valves. Device 100, including the valves and frame, may be any of various manufactured sizes. In percutaneous transcatheter A-V valve replacements, device 100 may be inserted via femoral vein access (or other large peripheral access vein) replacement. For transcatheter Mitral A-V valve replacements, combined trans-femoral (or other peripheral large access vein) and trans-septal access may be needed. Device 100 may be mounted and crimped on the delivery catheter such that after the native A-V valve is crossed in an antegrade direction, the ventricular end, which may be outflow skirt 118, may be deployed first, and the atrial end, which may be in-flow skirt 116, may be deployed last. The universal valve structure may be the same in FIGS. 3-4 with the only difference being conformance to physiologic blood flow direction based on position within the heart; however, the mounting, crimping and deployment may be flipped based on access in order to achieve the desired deployment position and required direction of flow. Depending on the valve, access site, route of deployment, and antegrade or retrograde direction of valve crossing, the mounting and deployment direction and unsheathing sequence for surgical or transcatheter use in both the V-A and A-V positions may be from a ventricular end first to either the arterial or atrial end. Use in the pulmonic position (surgical or transcatheter) may optionally use the opposite or reverse sequence (arterial to ventricular end). While the device mounting orientation and unsheathing deployment sequence may or may not vary for direct surgical insertion or trans-apical access, it would be understood that the in-flow skirt 116 would be the ventricular end in a V-A position and the in-flow skirt 116 would be the atrial end in an A-V position at the end of all deployments. For open heart surgical placement under direct vision for stenotic valves in either V-A or A-V positions, partial or full debridement of native diseased valvular or annular tissue may or may not be optionally performed prior to deployment.

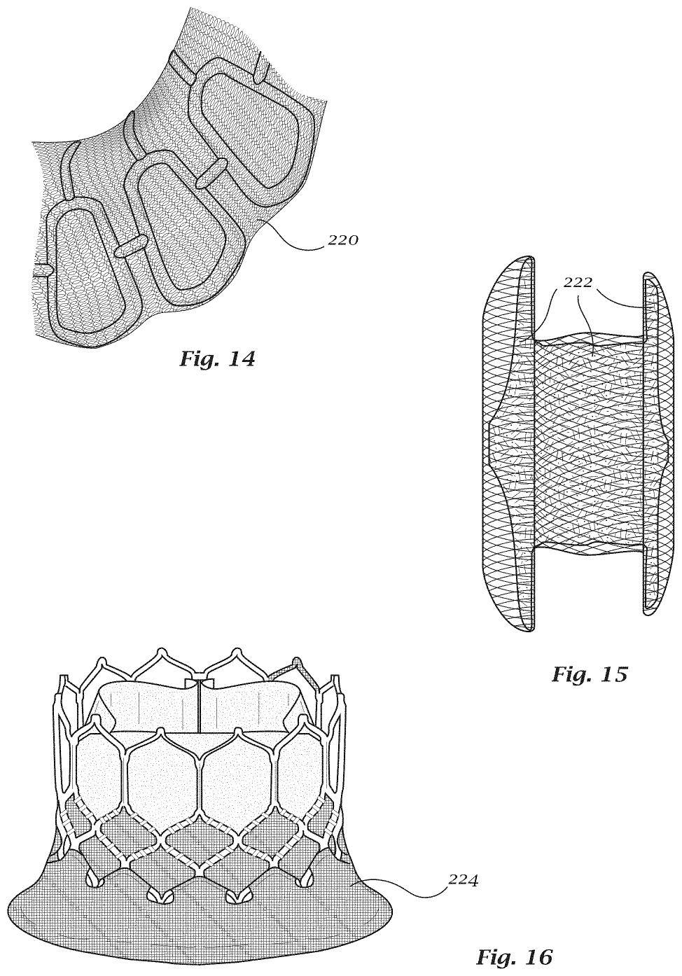

Now referring to exemplary FIGS. 5 and 14-16, a valve device 100 may be shown. Device 100 may be any of various manufactured sizes, including the valve and the frame. The flexible discs and radial/memory-shape forces may provide conformity enhancement for device 100. In addition, additional elements may be used to further enhance sealing. These elements may include biocompatible textile or biomaterial coverings (biomaterial attached to inner or outer frame), biocompatible textile lining (material between weave layers of flexible discs), or biocompatible textile coatings applied onto wire (i.e. integrated) with frame surfaces. These coverings, linings, and coatings may be applied in various combinations at any of the three core components of device 100, including inflow disc 116, central core 112, or outflow disc 118. For discs, coverings may be limited to non-tissue facing surfaces and may optionally be enhanced in thickness at an edge of the discs. Exemplary FIGS. 14-16 may show various underlying frame formations; however, the coating, lining, and covering embodiments may be employed with the device 100 as described according to various embodiments herein, including single layer or multi-layer woven wire formations, as shown and described. A coating 220 may be a biomaterial integrated with the frame surface, as shown in FIG. 14. A lining 222 may be a biomaterial placed in between layers of woven frame, as shown in FIG. 15. A covering 224 may be a separate biomaterial attached to outer tissue facing surfaces, as shown in FIG. 16. For the discs 116, 118, a coating 220 may be applied to or integrated with either or both discs and similarly a lining 222 may be integrated within or placed between both discs. For the central core 112, coatings 220 or coverings 224 may be applied to targeted areas of tissue facing surfaces, such as proximal to an inflow end, or may be applied anywhere along the entire length of the central core. Central core 112 may also be lined 222 if manufactured with multiple layers as a weave. The coatings, coverings or linings may be added as needed to maximize immediate sealing in order to minimize risk of paravalvular leaks, and to promote long-term healing and cellular ingrowth. According to some exemplary embodiments, an integral coating 220 may promote fixation and anchoring by providing a frictional component on tissue-facing surfaces. According to an exemplary embodiment, the cell size and design of central core 112 may be optimized for durability and valve washing. An open, closed or woven frame cell design with or without coatings and/or linings could be variably used to optimize any of the following: frame durability, fixation and sealing, conformity to native tissues, maintenance of round central core, washing of valve, hemodynamics and valve area. The size of device 10 central core 112 and central orifice 114 may vary. According to some embodiments, sizes may vary to accommodate valves 120 ranging from about 20 mm to about 34 mm internal diameter, which may be housed in the central orifice 114.

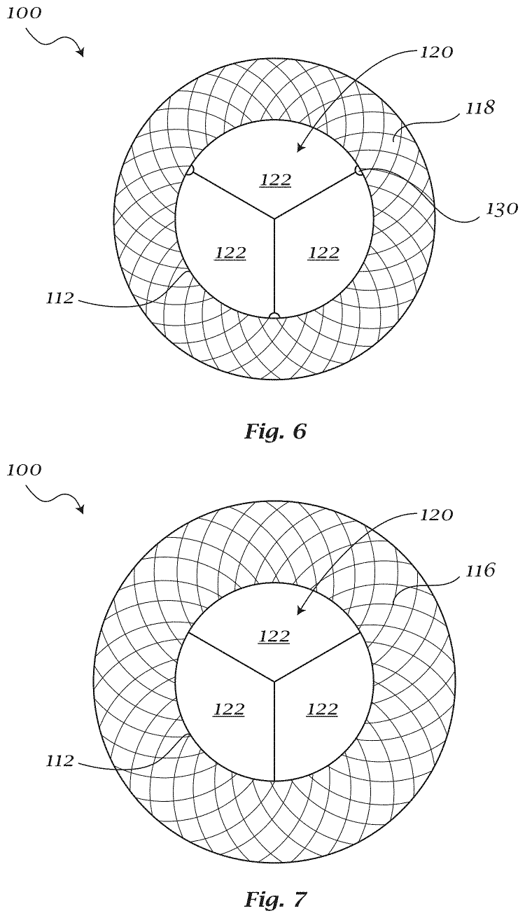

Now referring to exemplary FIG. 6, a valve device 100 may be shown from an outflow end. Valve device 100 may be shown from an in-flow end in FIG. 7. An exemplary replacement valve 120 may be a unidirectional trileaflet valve with three leaflets 122. A trileaflet embodiment may be utilized in all valve positions including the mitral, tricuspid, aortic, and pulmonary positions. Biologic, human or polymeric tissues or their derivatives may be used to construct the leaflets 122. Exemplary valve sizes, which may be the internal diameter of the central core 112, may be 20 mm, 23 mm, 25 mm, 27 mm, 29 mm, 31 mm, or 34 mm. Therefore, the internal diameter of the central core 112 may range from about 20 mm to about 34 mm, according to an exemplary embodiment. Furthermore, the external diameter of the outflow disc 118 may range from about 26 to about 57 mm. The outflow disc 118 may have a ratio of internal diameter/external diameter of about 0.65 to about 0.75. The external diameter of the inflow disc 116 may be about 31 mm to about 62 mm. The inflow disc 116 may have a ratio of internal diameter/external diameter of about 0.55 to about 0.65. The external diameters of the outflow disc 118 and inflow disc 116 may have a size where the outflow disc external diameter/inflow disc external diameter ratio is from about 0.65 to about 0.75. According to an exemplary embodiment, the valve height may equal the central core depth plus the depth of the inflow and outflow discs plus commissural post depth, and may have a valve height to valve size ration of about 0.75 to about 0.8. The outer diameter of the central core 112 may be about 1 to about 3 mm wider than the inner diameter.

Now referring to exemplary FIGS. 8-8A, valve device 100 may self-expand or be balloon expandable to a shape such that a patient's native valve and para-annular tissue 20 is gripped between the skirts 116, 118 and central core 112 of the frame 110. The radial expansion force of the central core 202 may be caused by one or more of the frame material properties (self-expanding memory shape) or by balloon expansion to a pre-determined shape. In-flow skirt 116 and outflow skirt 118 may be outwardly flexible 200 (away from a central transverse valve axis) to accommodate tissue and conform to anatomy while exerting a centrally directed or inward lateral force 201 (toward a central transverse valve axis) to allow optimal seating and anchoring. Optimal seating and anchoring may include pressure and/or frictional fixation, sealing and healing. In some embodiments, coatings and/or coverings may add to friction provided by tines 140. Furthermore, central core 112 may exert an outward radial force along with or parallel to the transverse valve axis or may optionally be balloon expandable to achieve a pre-determined diameter and round shape 200, as would be understood by a person having ordinary skill in the art. The diameter and length of central core 112 may be sized and positioned according to the level and dimensions of a patient's native valve and annulus, which may be determined using standard imaging and measurement techniques as would be understood by a person having ordinary skill in the art. As shown in FIG. 8A, arrows 200 may show the flexibility for inflow and outflow discs, arrows 201 may show the direction of memory shaped forces and arrows 202 may show the direction of radial forces along a transverse valve axis.

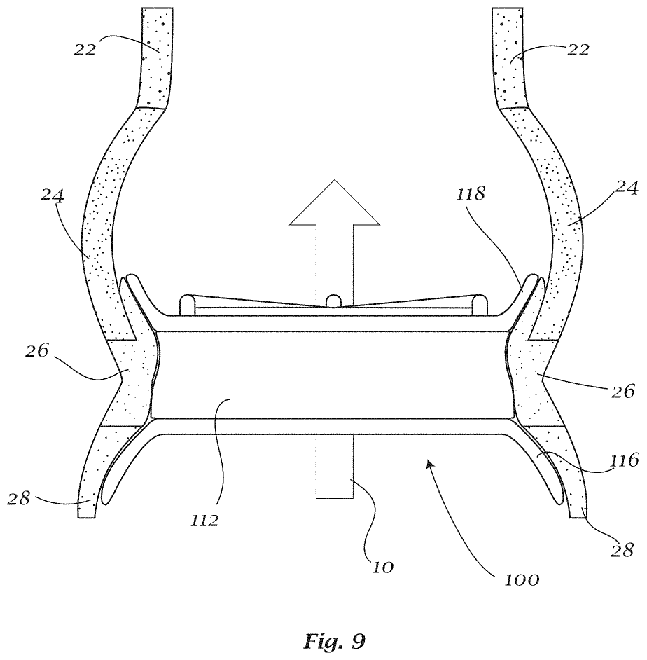

As shown in exemplary FIG. 9, a deployed valve device 100 may be deployed in a Ventriculo-Aortic valve position, such as the Aortic position. In the Aortic position, blood may flow from the left ventricular outflow tract 28 through a replacement valve 120 of device 100, which may open in systole, and into the sinus of Valsalva 24 toward the ascending aorta 22. Device 100 may be deployed such that central core 112 self-expands or is dilated to align in the direction of flow at the level of the native annulus and super-annular valve tissue 26. The flexible in-flow skirt 116 may expand or be dilated to grip one or more of the left ventricular outflow tract 28 and/or the annulus 26. Similarly, outflow skirt 118 may expand or be dilated to grip one or more of the annulus and valve tissue 26 and the proximal tissue of the sinus of Valsalva 24, avoiding distortion or occlusion of the orifices of the right or left main coronary arteries. The native annulus and valve tissue 26 may be forced outward, away from the central orifice 114, by the outflow skirt 118 and/or central core 112 and may be reflected toward or pinched between the outflow skirt 118 and/or central core 112 and the proximal wall of the Sinus of Valsalva 24. Outflow skirt 118 may optionally extend beyond the existing annulus and valve tissue 26, depending on the position of the proximal coronary arteries, forming a small sub-coronary rim above residual compressed native valve tissue. For pulmonary valve implants, coronary artery position would not be a concern.

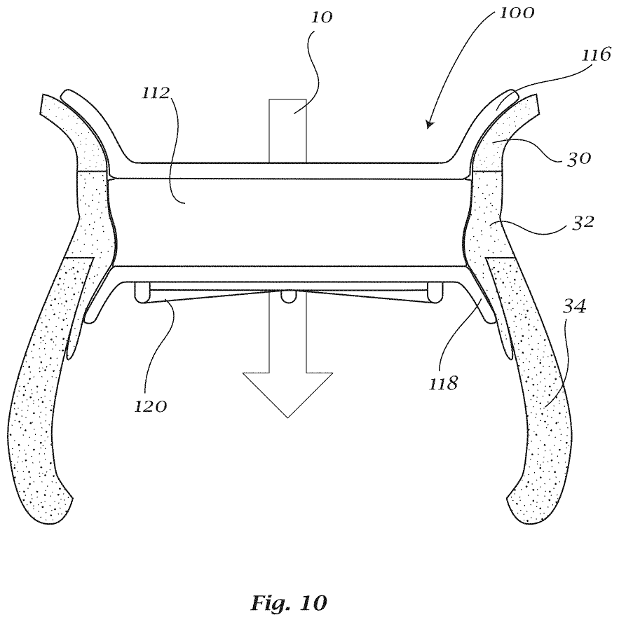

As shown in exemplary FIG. 10, a deployed valve device 100 may be deployed in an Atrio-Ventricular valve position, such as the Mitral position. In the Mitral position, blood may flow from the atrium 30 through a replacement valve 120 of device 100, which may open in diastole, and into the ventricle 34. Device 100 may be deployed such that central core 112 aligns in the direction of flow at the level of the native annulus and sub-annular valve tissue 32. In-flow skirt 116 may expand to conform to the atrial tissue 30 proximal to the annulus. Central core 112 and outflow skirt 118 may expand to grip one or more of the annulus and valve tissue 32 and/or the ventricle wall 34. The native annulus and valve tissue 32 may be forced outward, away from the valve central orifice 114, by the central core 112 and outflow skirt 118 and may or may not be pinched between the outflow skirt 118 and the wall of the ventricle 34. Outflow skirt 118 may optionally extend beyond the existing annulus and valve tissue 32. The length and force of the outflow skirt 118 may be optimized by the design of frame 110 to prevent or minimize the risk of causing systolic anterior deviation of the anterior mitral leaflet that may create obstruction of the left ventricular outflow tract.

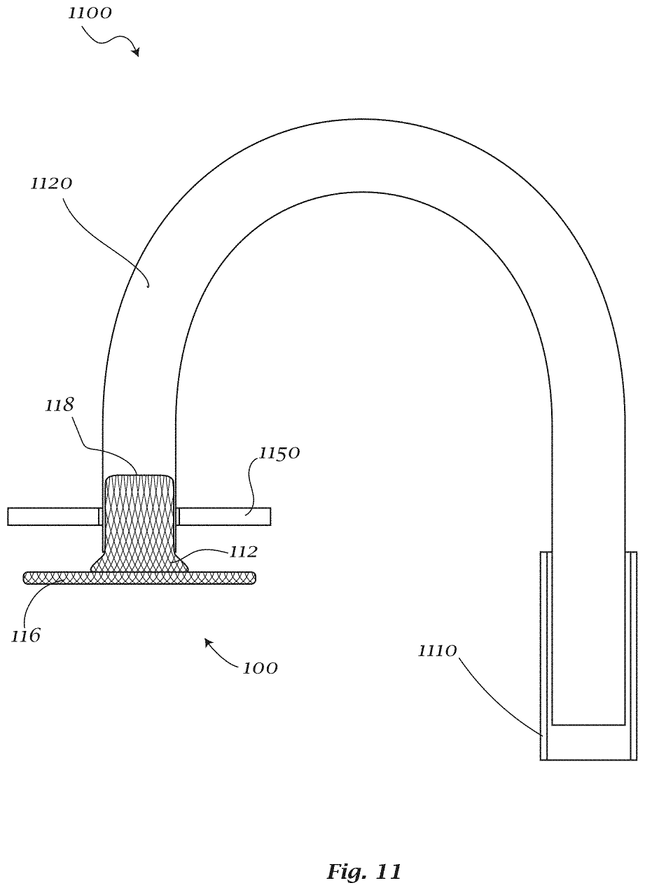

Now referring to exemplary FIG. 11, a steerable transcatheter V-A valve arterial introducer system 1100 for retrograde percutaneous transcatheter replacement of the aortic valve may be shown. As shown, an outer introducer sheath 1110 may provide central access via the femoral artery for AV or femoral vein for PV replacement. An inner steerable trans-femoral V-A valve deployment catheter 1120 may be inserted through sheath 1110 using a femoral arterial approach to allow retrograde crossing of the native aortic valve (shown in FIG. 11). Alternatively, an antegrade crossing of the V-A valve may be required when using transapical access (aortic valve) or for femoral venous approach to the pulmonic valve (neither being shown in the Figures). In addition, other access routes to the respective V-A valves may be used if blockages of the primary access routes are present or if surgical access is used. The typical aortic valve orientation of the device 112 within the steerable deployment catheter 118 may be an exemplary orientation for uses where the native diseased V-A valve is crossed in a retrograde fashion, as shown in FIG. 11. The device may be steered to align with the native aortic annulus and valve tissue 1150. To achieve the desired aortic valve orientation (FIG. 9), the universal valve device 100 may be crimped (collapsed) and loaded in the deployment catheter 1120, such that the ventricular end of in-flow skirt disc 116 is unsheathed or deployed first (retrograde approach and deployment). Gentle traction may help anchor the skirt disc 116 on sub-annular tissue of the native V-A valve outflow tract with the disc parallel to the aortic valve plane. According to some embodiments, the anchoring may be augmented by the memory shape of the frame and/or tines disposed on the frame. The remaining central core 112 with replacement valve 120 and arterial outflow skirt 118 may initially remain inside the deployment catheter. Continued unsheathing of the device 112 from within the deployment catheter 118, which may result from additional traction or from an active deployment mechanism, may result in sequential deployment of the remaining central core 112, valve 120, and remaining arterial side outflow skirt disc 118. A brief period of rapid ventricular pacing may facilitate accurate device placement. Pre-dilation (i.e. balloon valvuloplasty) may also beoptionally be used. The full device 100 may be retrievable or re-sheathable prior to final release of the device 100 from the introducer system 1100.

Some embodiments using a balloon expandable version may require a full unsheathing and balloon expansion under rapid ventricular pacing to allow attainment of the final pre-determined shape. For some uses and embodiments (e.g. transapical aortic valve or transvenous pulmonary valve), antegrade crossing the native V-A valve may need alternative mounting, crimping and deployment sequences. For access approaches using antegrade crossing of the native V-A valve, the valve 100 may be crimped and loaded in the steerable introducer sheath 1120 in the opposite orientation such that the outflow disc 118 is deployed first. With gentle downward traction on the deployment sheath, or by an active deployment mechanism, sequential deployment of the central core 112 and the inflow disc 116 may follow. Open surgical V-A valve replacement with access under direct vision (via aortotomy for AV; via Pulmonary arteriotomy for PV) may use retrograde V-A valve crossing and deployment techniques and may use shorter introducer sheaths designed specifically for this purpose. Depending on the disease and local anatomy, open heart direct vision surgical access to the valve may or may not be accompanied by limited resection or debridement of diseased annular or valvular tissue provided sufficient tissue for anchoring is left in place as would be understood by a person having ordinary skill in the art.

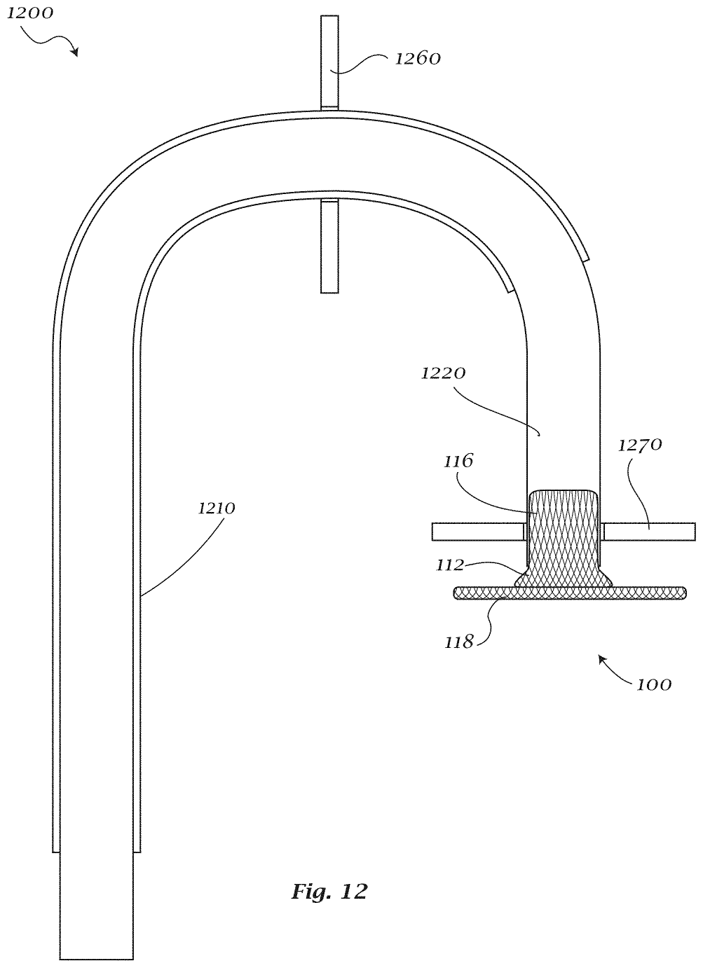

Now referring to exemplary FIG. 12, a transcatheter A-V valve introducer system 1200 for mitral valve replacement may be shown. As shown, an outer steerable A-V valve transfemoral vein introducer sheath 1210 may be provided. An inner steerable valve deployment catheter 1220 may be used to hold and steer device 100. Addition of trans-septal access 1260 may be used for transcatheter mitral valve replacement as shown in FIG. 12. The steerable introducer sheath 1210 may be used to guide the deployment catheter 1220 through the septum 1260, as would be understood by a person having ordinary skill in the art. Once access is obtained, the steerable deployment catheter 1220 may be used to align the device 100 with the native mitral or tricuspid annulus and valve tissue 1270 after crossing the valve in an antegrade direction, as would be understood by a person having ordinary skill in the art. In all A-V valve deployments, whether surgical or transcatheter, antegrade crossing of the valve may be employed, allowing for uniform crimping and loading of the device 100 in all A-V valve uses. The universal valve device 100 may be crimped and loaded in the deployment catheter 1220 such that the ventricular end outflow skirt 118 is unsheathed or deployed first. Traction may anchor the outflow skirt 118 on sub-annular valvular tissue of the native A-V valve complex. According to some embodiments, the traction may be caused by the expanding force of the frame and/or tines disposed on the frame. Retraction of the deployment sheath 1220 may be active (gentle traction) or passive (mechanical action) and may sequentially unsheathe the outflow skirt 118, the remaining central core 112 with valve 120, and atrial side in-flow skirt 116. The full valve device 100 may be retrievable or re-sheathable prior to final release or deployment of the distal central core 112 and valve 120 from the introducer system 1200. For surgical deployments, A-V valves may be replaced under direct vision (open heart access) or image guided indirect vision (closed heart per-atrial surgical access) using shortened modified deployment catheters that employ using similar techniques as those described above and as would be understood by a person having ordinary skill in the art. For the mitral position, the steerable trans-septal access and deployment catheters may use antegrade transfemoral vein access. For tricuspid position, the steerable deployment catheter may also travel in an antegrade direction via a large peripheral access vein (e.g. femoral, subclavian vein or internal jugular veins) but without the need for trans-septal puncture. Should unusual circumstances require occasional trans-apical access to the mitral valve, a reverse mounting and unsheathing sequence (atrial to ventricular end) may be used.

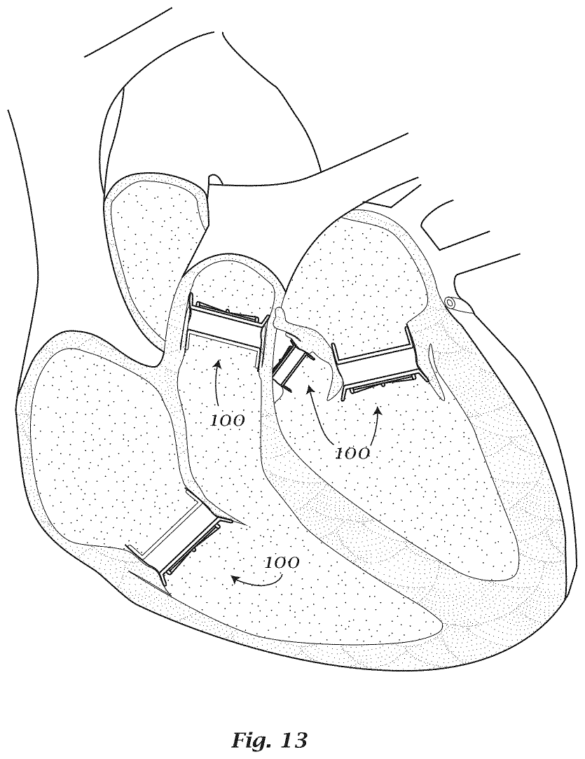

Now referring to exemplary FIG. 13, the universal nature of the variably sized and oriented device 100 may be possible from the unique frame design. The design includes a memory shaped open and woven cell design, where the device has a rigid central core (open or closed cell), when deployed, housing a unidirectional tricuspid valve, with flexible (woven cell) inflow and outflow discs. The design further includes unique anchoring characteristics, namely, three level rebound memory pressure-based anchoring. This anchoring uses radial memory forces perpendicular to the longitudinal axis of blood flow (i.e. exerted parallel to the transverse axis of a valve) applied by the central core against peri-annular and valvular tissues. Rebound memory forces may be applied laterally and centrally toward the transverse axis of the valve by the flexible inflow disc. Rebound memory forces may also be applied laterally and centrally toward the transverse axis of the valve by the flexible outflow disc. Three level frictional anchoring, including a plurality of tines disposed on tissue facing surfaces of the central core, inflow disc and outflow disc, may augment the rebound memory pressure-based anchoring. Multilevel and multimodality anchoring may allow the device to be used for any variety of heart valve pathology. Device 100 may further be capable of enhanced sealing and healing characteristics to minimize risks of paravalvular leak. These characteristics may be enhanced by three level coating, lining and/or covering of valve frame structures in various combinations to promote immediate sealing and long-term healing. The woven frame design of the flexible outflow disc may allow biologic or biologically compatible linings to be woven between layers or surface coatings to be applied to the disc. Furthermore, the closed or open cell design of the central core may allow application of biologic or biologically compatible materials to be integrated with the device, in coating embodiments, or attached to the device, in covering embodiments. Lastly, the universal nature may be facilitated by the unique deployment system. Specially designed deployment catheters may allow for an accurate final placement position oriented correctly for location, alignment, centeredness, and the direction of desired flow regardless of access site method or route. Deployment catheters may allow mounting, loading and crimping of the device valve in the direction appropriate for the location of use and the direction in which the native valve is crossed, antegrade or retrograde. Deployment catheters may also employ a passive (traction) or active (expulsion) mechanism to cause valve exit from the deployment catheter, as would be understood by a person having ordinary skill in the art. Deployment catheters of different lengths may also facilitate ease of use as dictated by needs for alternative access routes and uses.

The universal heart valve device, including embodiments described herein, may allow for the properly sized valve device to be flipped in orientation for loading, mounting, and crimping on a deployment catheter in the proper direction needed for achieving final deployment with secure anchoring and sealing in the correct anatomic position and physiologic valve orientation required for proper functioning of the replacement valve at any native heart valve site (i.e. aortic, mitral, pulmonic, tricuspid) or any combination of sites based on methods and routes of access. The device may further allow and promote conformity, anchoring, fixation and sealing to occur at up to 3 levels of the valve device (i.e. inflow disc, central core, and outflow disc) in a full range of anatomically and physiologically appropriate sizes so that forces, which may include rebound memory and radial forces, exerted by the frame may provide correct self-centering and self-aligning positioning, appropriate physiologic orientation and function, and secure anchoring, fixation and sealing of the heart valve device within any treated native heart valve and its paravalvular tissues. The device may further allow and promote conformity, anchoring, fixation and sealing at up to 3 levels of the valve device for any native valve site of insertion regardless of the type or severity of underlying valvular or annular pathology. Furthermore, the device may allow and promote conformity, anchoring, fixation and sealing at up to 3 levels of the valve device for any native valve site of insertion regardless of the method of access. The device may also allow and promote conformity, anchoring, fixation and sealing at up to 3 levels of the valve device for any native valve site of insertion regardless of the route of access. The device may allow and promote self-centering deployment, positioning and orientation that is anatomically and physiologically correct at any of 4 native heart valve locations within the heart. This may be achieved regardless of the site, method, or route of peripheral, central, or direct access using any combination of transcatheter or surgical techniques and may include a plurality of sites. This may also be achieved regardless of direction in which the native heart valve is crossed (i.e. antegrade or retrograde) during deployment. The design, anchoring, fixation, sealing, and conformity mechanisms of the device, which may be provided by the design, materials, and sizing, may allow the valve to be used regardless of the underlying native valve pathology in any of the native heart valves. Finally, the design, anchoring, fixation, sealing, and conformity mechanisms may reduce or prevent the risk of paravalvular leak regardless of the native heart valve position where it is used.

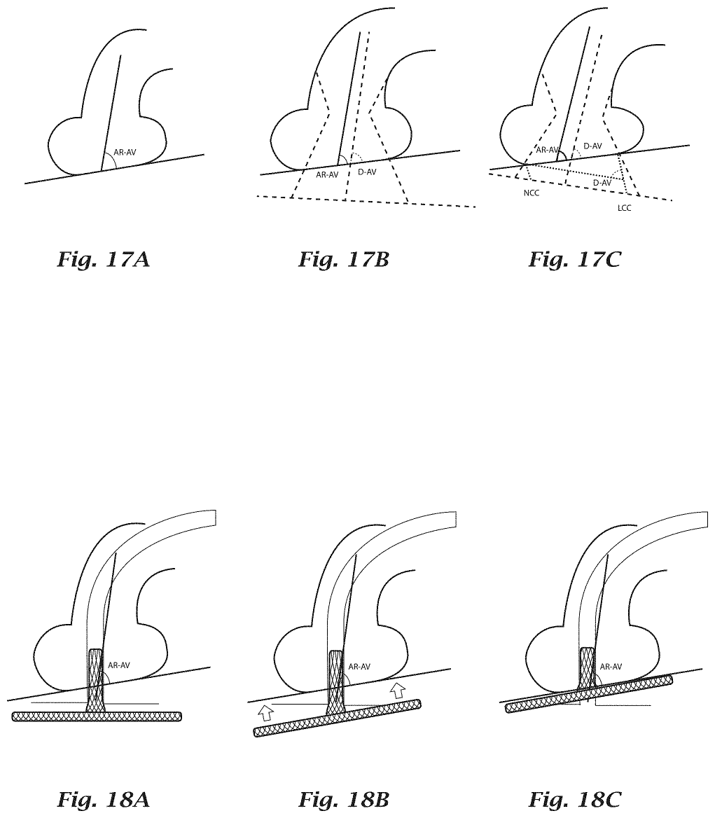

Now referring to exemplary FIGS. 17A-17C, a typical deployment of conventional self-expanding valves may be shown in an aortic position. Conventional valves (both self-expanding and balloon dilated transcatheter valves), which, after deployment, have a rigid frame throughout their length, may be prone to paravalvular leak, deployment angulation and seating issues (i.e. deployment that is too high or too low) caused in part by the rapid expansile forces exerted by conventional transcatheter valves and various degrees of angulation between the longitudinal axis of the aorta and aortic root and the aortic annular plane (AR-AV). These issues may present themselves in any insertion location. The longitudinal axis of a fully deployed conventional transcatheter device may deploy parallel to the longitudinal axis of the aorta or aortic root. The angle between the device longitudinal axis and aortic valve plane (D-AV) may be equal to the angle between the aortic root axis and the aortic valve plane (AR-AV). Therefore, the device may project beyond the aortic valve plane at an angle, such that, depending on deployment depth, a gap may exist between the device frame and the aortic valve plane. As shown, an inflow end of a conventional device frame may be angled toward the valve plane on a non-coronary cusp (NCC) side and angled away from a valve plane on a left coronary cusp (LCC) side. The gap between the native valve plane and the LCC side of the device may allow for paravalvular leak. Depending on the direction and degree of angulation of the aorta and aortic root in relation to the annular plane, angular mal-placement of the conventional device may result at any point along the circumference of the conventional transcatheter device resulting in gaps between the device and tissues to which it is anchoring. Variations in depth of deployment of conventional devices resulting from rapid expansile forces may exacerbate these gaps. To the contrary, the inflow and outflow discs of the material memory and flexibility of the universal valve device and the method of deployment may promote flexible disc conformity to the native structure and physiology despite various angulation differences of the native anatomy, as shown in FIGS. 18A-18C. The inflow and or outflow disc may (depending on site of deployment and direction of native valve crossing during deployment) conform to the native structure anatomically adjacent to the annular plane as a result of the flexible design of the disc and the staged method of deployment. As shown in FIG. 18B, the deployed leading flexible disc (i.e. flexible inflow disc for aortic valve position with retrograde valve crossing) of the partially deployed universal heart valve in the aortic position may, when traction is applied to the deployment catheter, tilt to self-align, self-center and conform with the sub valvular tissue just below the level of the aortic valve annular plane. In FIG. 18C, the inflow disc may reach its final position, prior to deployment of the central core and remaining flexible disc, where it may be self-aligned, self-centered, parallel and conforming to the annular plane. By flexibly conforming to the tissue adjacent to the annular plane prior to the deployment of the remaining valve (i.e. central core and outflow disc in depicted example), optimal full valve deployment at the correct height (i.e. neither too high or too low) where the valve opening is aligned and parallel to the longitudinal axis of blood flow and the central core frame is seated and centered parallel to the annular plane is encouraged. Such deployment may eliminate or reduce gaps caused by anatomic and or deployment catheter angulation relative to the annular plane and may result in radial pressure necessary for fixation at any site to be reliably applied to annular and valve tissues distal to the level of the annular plane where conduction tissues are least likely to be affected. This may further prevent or reduce paravalvular leak by preventing or minimizing blood flow via gaps between the discs and the native tissue, and may reduce or eliminate the need for new pacemaker related to conduction tissue injury resulting from pressure forces exerted by low or mis-aligned valve deployment of conventional self-expanding and balloon-dilated transcatheter valves.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A sutureless universal heart valve device comprising:
a frame having an inflow disc, a central core defining a central orifice, and an outflow disc, wherein the frame is self-expanding or balloon dilatable;
a plurality of tines disposed on the frame for engaging native tissue, wherein the plurality of tines are disposed on native tissue facing surfaces of the frame; and
a prosthetic valve housed in the central orifice, wherein the prosthetic valve is a one-way valve and comprises bioprosthetic or polymeric materials,
wherein the central core is radially compressible and self-expanding memory-shaped wire or balloon expandable open cell wire,
wherein the inflow and outflow discs are compressible and flexible memory-shaped woven wire or balloon expandable open cell wire,
wherein the inflow disc has a larger diameter than the outflow disc, and
wherein the inflow disc, central core, and outflow disc are configured to exert radial and memory shape forces to conform, compress, and grip native heart valve and paravalvular tissues for fixation and anchoring.

2. The device of claim 1, wherein the plurality of tines project from at least one of the inflow disc, central core, and outflow disc only on surfaces adjacent to native tissue.

3. The device of claim 1, further comprising a coating deployed on at least a portion of the frame, wherein the coating promotes at least one of sealing and long-term healing.

4. The device of claim 1, further comprising a lining secured within layers of the woven wire of at least one of the inflow disc, the outflow disc, and the central core, wherein the lining promotes at least one of sealing and long-term healing.

5. The device of claim 1, further comprising a covering disposed on a surface of at least one of the central core, the inflow disc and the outflow disc, wherein the covering promotes at least one of sealing and long-term healing.

6. The device of claim 1, further comprising a plurality of commissure posts for securing the prosthetic valve leaflets to the frame.

7. The device of claim 1, wherein the prosthetic valve is a uni-direction, tri-leaflet valve.

8. The device of claim 7, further comprising three commissure posts for securing the prosthetic valve to the frame, wherein each valve leaflet is secured to two commissure posts.

9. The device of claim 1, wherein the inflow disc, central core, and outflow disc are configured to be sequentially deployed; and wherein the inflow disc, central core, and outflow disc exert radial and memory shape forces to conform, self-align and self-center parallel to an annular plane within any native annular heart valve and paravalvular tissues.

10. A method of deploying a universal heart valve device comprising:
determining a position for the device within a native heart valve;
determining a method of access to the native heart valve to be replaced;
determining a route of access to be used for deployment of the heart valve device;
measuring a native valve annulus and paravalvular dimensions;
selecting a device having appropriate length, diameter, and valve size for the native heart valve to be replaced, wherein the device comprises:
a frame having an inflow disc, a central core defining a central orifice, and an outflow disc;
a plurality of tines disposed on the frame for engaging native tissue;
and a prosthetic valve housed in the central orifice,
wherein the central core is radially compressible and self-expanding memory-shaped wire,
wherein the inflow and outflow discs are compressible and flexible memory-shaped woven wire,
wherein the inflow disc has a larger diameter than the outflow disc, and
wherein the inflow disc, central core, and outflow disc are configured to compress and grip native tissue of a heart;
mounting, loading and crimping the device in a steerable deployment catheter in a direction appropriate for the location of use and the direction in which the valve is crossed, being antegrade or retrograde;
steering the device within the deployment catheter to align with the native annulus and valve tissue;
positioning the device and deploying the inflow disc or outflow disc depending on the site and direction of access;
applying traction to the deployment catheter to promote conforming, self-alignment and self-centering of the deployed disc parallel and proximal to an annular plane;
deploying the central core and prosthetic valve; and
deploying the remaining inflow or outflow disc.

11. The method of claim 9, wherein the device is loaded for A-V positions such that the inflow disc is deployed first.

12. The method of claim 9, wherein the device is loaded for V-A positions, such that the outflow disc is deployed first.

13. The method of claim 9, wherein the method of access includes at least one of transcatheter, open heart surgical, and closed heart surgical methods of access.

14. The method of claim 9, wherein the routes of access include at least one of percutaneous, direct vessel exposure, purse strings, hemostatic access, and direct exposure of the native heart valve during open heart surgery.

15. The method of claim 13, wherein the percutaneous or direct vessel exposure routes comprise at least one of peripheral arterial access, peripheral venous access, large central artery access and central vein access.

16. The method of claim 13, wherein the purse strings or hemostatic access routes comprise at least one of direct per-atrial artery access, direct per-ventricular artery access, direct per-aortic artery access, and direct per pulmonary artery access.
